(19) United States
(12) Patent Application Publication
(10) Pub. No.: US 2026/0272796 A1
(43) Pub. Date: Sep. 17, 2026

(54) Transparent and Mild Surfactant Wash Composition

(71) Applicant: Conopco, Inc., d/b/a UNILEVER, Hoboken, NJ (US)

(72) Inventors: Michelle Mary FALLER, Fairfield, CT (US); Bivash Ranjan Dasgupta, Cheshire, CT (US)

(73) Assignee: Conopco, Inc., d/b/a UNILEVER, Hoboken, NJ (US)

(21) Appl. No.: 19/077,521

(22) Filed: Mar. 12, 2025

(51) Int. Cl. A61K 8/365; A61Q 5/02; A61Q 19/10

(52) U.S. Cl. CPC A61K 8/365; A61Q 5/02; A61Q 19/10; A61K 2800/262; A61K 2800/596

(57) ABSTRACT

The present invention is directed to a transparent wash composition comprising a PPAR agonist. The wash composition is stable, pumpable, substantially free of sulfate-based surfactant, and remains transparent with high levels of PPAR agonist.

### FIELD OF THE INVENTION

The present invention is directed to a transparent and mild surfactant wash composition. More particularly, the composition comprises 18% by weight or less total surfactant. The wash composition is surprisingly transparent even when formulated with as much as 2% by weight of an activator of a peroxisome proliferator activated receptor. The wash composition is stable, lathers and rinses well even with the activator of a peroxisome proliferator activated receptor included. Such a composition can be formulated substantially free of at least one of a sulfate-based surfactant, paraben, hydantoin, isothiazolinone, phthalate, acrylate-based thickener, soap, dioxane and silicone oil.

### BACKGROUND OF THE INVENTION

Aqueous wash compositions, such as detergents, face washes, shampoos, body and hand washes are staples for many consumers. With consumers washing more, especially post pandemic, they demand that the wash compositions they use not only clean well but also deliver exceptional benefits to their skin. Additionally, certain consumers enjoy clear or transparent washes, like body washes, since a transparent appearance is generally associated with a perceived pure and gentle composition that is mild to skin and hair.

Unfortunately, when formulating transparent wash compositions, it is not routine to include skin benefit agents that are fatty in nature, and especially, those with hydrophobic portions having C₁₆ or greater linear chains including those which are activators of a peroxisome proliferator activated receptor. Fatty components often tend to drop or precipitate out of aqueous wash compositions, especially water continuous wash compositions, yielding a composition that gels or creams and that is opaque in appearance.

It is of increasing interest to develop wash compositions that are transparent but that do comprise fat-based skin benefit agents. The present invention, therefore, is directed to a transparent and mild surfactant wash composition that comprises 18% by weight or less total surfactant. The wash composition is surprisingly transparent with a viscosity from 1 to 1,100 m Pa-s even when formulated with as much as 2% by weight of an activator of a peroxisome proliferator activated receptor. The wash composition is stable, and lathers and rinses well even with the activator of a peroxisome proliferator activated receptor present. Such a composition is pumpable through a foam dispensing pump and can be formulated substantially free of at least one of a sulfate-based surfactant, paraben, hydantoin, isothiazolinone, phthalate, soap, dioxane and silicone oil.

### Additional Information

Efforts have been disclosed for making wash compositions. In U.S. Pat. No. 10,376,456, concentrated and self-preserving compositions of mild surfactants for personal care applications are described.

Other efforts have been disclosed for making wash compositions. In U.S. Patent Application Publication No. 2023/0414468 A1, a wash composition that does not irritate the eyes and that has anionic surfactant, amphoteric or zwitterionic surfactant or both, and nonionic surfactant or cationic polymer or a mixture thereof is described.

Further efforts have been disclosed for making wash compositions. In U.S. Pat. No. 8,148,309, wash compositions with transparency and acrylic polymers are disclosed.

Even other efforts have been disclosed for making wash compositions. In U.S. Pat. No. 5,902,779, substantially transparent cleansers comprising solvent, fatty acid surfactant and behenamidopropyl PG-dimonium chloride are described.

Still other efforts have been disclosed for making wash compositions. In U.S. Pat. No. 11,000,462 B2, wash compositions that can include fatty acid, fatty acid soap and 12-hydroxystearic acid are described.

None of the additional information describes a transparent wash composition that comprises an activator of a peroxisome proliferator activated receptor as claimed herein.

### SUMMARY OF THE INVENTION

In a first aspect the present invention is directed to a wash composition comprising:
a) from 4 to 18% by weight total surfactant, the surfactant comprising anionic surfactant, and amphoteric surfactant wherein 50 to 90% by weight of total surfactant in the wash composition is the amphoteric surfactant;
b) preservative;
c) from 0.1 to 2% by weight an activator of a peroxisome proliferator activated receptor;
d) alcohol, and
e) water,
   wherein the composition:
i) is transparent;
ii) has a pH from 5.65 to 8.5, and preferably, 6 to 8, and most preferably, from 6.2 to 7.8 or from 6.3 to 7.6, or from 6.4 to 7.5, or from 6.5 to 7.4; and
iii) comprises less than 0.065%, and preferably, less than 0.062%, and most preferably, less than 0.06%, by weight or less ethylene glycol dilaurate based on total weight of the wash composition,
   the amphoteric surfactant comprises 25% or less, and preferably, 18% or less, and most preferably, 10% or less, or from 0.0 to 8%, or from 0.01 to 4%, or 0.0% by weight hydrophobic portion derived from triglyceride based on total weight of hydrophobic portion present on the amphoteric surfactant.

In a second aspect, the present invention is directed to a method for washing a surface, like skin or hair, with the wash composition of the first aspect of the invention.

In a third aspect, the present invention is directed to the use of an anionic surfactant and amphoteric surfactant to solubilize an activator of a peroxisome proliferator activated receptor in a wash composition substantially free of a sulfate-based surfactant and comprising less than 0.065% by weight ethylene glycol dilaurate.

All other aspects of the present invention will more readily become apparent from the description and Examples which follow.

Skin, as used herein, is meant to include skin on the arms (including underarms), face, back, feet, neck, chest, hands, legs, buttocks and scalp. Stable means the wash composition displays no syneresis and remains transparent or clear for (3) weeks, and preferably, for one (1) month, and most preferably, for two (2) months after being stored at 22° C. and as determined by visual inspection from trained panelists. Viscosity values used herein are measured at 25° C. with a Discovery HR-2 Rheometer using sand blasted plates having a 1000-micron gap and a shear rate of 10⁻¹ s⁻¹. Lathers and rinses well means having a lather or foam volume and wash off or rinse characteristics consistent with more traditional compositions having no more than 18% by weight total surfactant and no activator of a peroxisome proliferator activated receptor formulated therein as determined by skilled panelists washing with water at about 37° C. with wash composition and water at a weight ratio from 1:6 to 1:3 or from 1:5 to 1:4.5 or from 1:4.5 to 1:4.

Isotropic means a composition with micelles arranged without directional preference. The wash composition of the present invention is preferably isotropic. Soap, as used herein, means salt of a fatty acid. Amphoteric surfactant means a surfactant that changes from positive or cationic to negative or anionic as the pH goes from acidic to basic wherein the term amphoteric as used herein is meant to include zwitterionic surfactants, surfactants that can simultaneously maintain both a cationic and anionic charge at a certain pH.

The wash composition of the present invention is one which surprisingly has a viscosity of at least 1, and preferably, at least 5, and most preferably, at least 8 mPa-s where viscosity is taken as previously defined. The wash composition of this invention is one suitable to be wiped or washed off, and preferably, washed off with water. Such a composition can be a home care cleaning composition (e.g., for tabletops, glass, floors, toilets, dishes, upholstery, or laundry) but is preferably a shampoo, make-up wash, facial wash, hand wash, or personal care and liquid body wash. In an embodiment of the invention, the wash composition can have a viscosity from 1 to 1,100 mPa-s, and preferably, and most preferably, from 5 to 1,000 mPa-s, or from 6 to 600 mPa-s, or from, or 6 to 500 mPa-s, or from 7 to 400 mPa-s, or from 7 to 300 mPa-s, or from 7 to 250 mPa-s, or from 8 to 200 mPa-s, or from 8 to 50 mPa-s, or from 8 to 35 mPa-s, or from 1 to 45 mPa-s, or from 1 to 40 mPa-s or from 1 to 38 mPa-s. When used for hand applications, the wash composition of the present invention will preferably have a viscosity that is at least 5 mPa-s and often not higher than 900 mPa-s.

The wash composition of this invention may, optionally, comprise medicinal or therapeutic agents, but preferably, is a wash which is for cosmetic and non-therapeutic uses to remove dirt, oil, make-up or the like from surfaces, especially the skin and scalp. In a preferred embodiment, the wash composition is a personal wash composition, and therefore, a liquid face or body wash for use while showering or bathing. Such composition may optionally comprise skin benefit ingredients added thereto such as emollients, vitamins and/or derivatives thereof, resorcinols, retinoic acid precursors, colorants, moisturizers, sunscreens, antibacterial agents, ceramides, mixtures thereof or the like. Such skin benefit ingredients (or agents) can be water and/or oil soluble. If used, oil soluble skin benefit agents typically make up to 2% (or up to 1.5%) by weight of the wash composition with the proviso that the activator of a peroxisome-proliferator-activated receptor (“PPAR agonist”) is meant to be independent from the optional oil soluble skin benefit agent that may be used. Water-soluble skin benefit agents, when optionally used, may make up to 10% (or up to 5%) by weight of the wash composition. The wash composition will typically have a pH 5.65 to 8.5, and preferably, 6 to 8, and most preferably, from 6.2 to 7.8 or from 6.3 to 7.6, or from 6.4 to 7.5, or from 6.5 to 7.4. PPAR agonists means compounds that activate receptors identified as peroxisome-proliferator-activated receptors; and particularly, a lipid that activates the nuclear receptor PPAR, and particularly, alpha. Transparent as used herein is synonymous with clear and means free of cloudiness or haziness as determined by visual inspection of trained panelists, and includes compositions that allow light to pass through with no diffusion to no more than 12% or no more than 10% or no more than 8% of the light provided being diffused.

As used herein, “substantially free of” as it relates to sulfate-based surfactants including ethoxylated sulfate-based surfactants (e.g., sodium lauryl sulfate or sodium pareth sulfate) means less than 1%, and preferably, less than 0.8%, and most preferably, less than 0.5%, even more preferably, less than 0.2% or less than 0.1% by weight of the wash composition. In an embodiment of the invention, “substantially free of sulfate-based surfactant includes a wash composition with 0.0% (i.e., none) by weight sulfate-based surfactant. Regarding the non-sulfate-based ingredients (i.e., ingredients, like a hydantoin preservative, phthalate, polymeric thickener, polymeric suspending agent), substantially free of means less than 0.6% by weight, and preferably, less than 0.4%, and most preferably, less than 0.3%, (or less than 0.22% or less than 0.1% or less than 0.05%) by weight of the wash composition of the invention. As to the non-sulfate-based ingredients, substantially free of also includes 0.0% (i.e., none) by weight of the ingredient in the wash composition of the invention. As to soap (e.g., sodium stearate or sodium oleate), the wash composition of this invention can have less than 2%, and preferably, less than 1%, and most preferably, less than 0.75% by weight soap, or less than 0.55%, or less than 0.25%, or from 0.001 to 0.24% or 0.0% (no soap) by weight soap based on total weight of the wash composition. With respect to all ingredients that the wash composition of the present invention may be substantially free of, it is within the scope of the invention to include from 0.001 to 0.045% by weight of such ingredient. In yet another preferred embodiment, the wash composition of the present invention comprises less than 35 ppm, and preferably, less than 25 ppm, and most preferably, less than 15 ppm dioxane or less than 2 ppm or less than 1 ppm dioxane or 0.0 ppm dioxane based on total weight of dioxane in the wash composition. In another embodiment, the wash composition of this invention may comprise from 0.00001 to 0.00005% by weight dioxane, like 1,4-dioxane.

The term comprising is meant to encompass the terms consisting essentially of and consisting of. For the avoidance of doubt, and for illustration, the wash composition of this invention comprising anionic surfactant, amphoteric surfactant and PPAR agonist is meant to include a composition consisting essentially of the same and a composition consisting of the same. All ranges defined herein are meant to include all ranges subsumed therein. Except in the operating and comparative examples, or where otherwise explicitly indicated, all numbers in this description indicating amounts or ratios of materials or conditions and/or physical properties of materials and/or use are to be understood as modified by the word “about”. The disclosure, as found herein, is to be considered to cover all embodiments as found in the claims as being multiply dependent upon each other irrespective of the fact that claims may be found without multiple dependency or redundancy.

### DETAILED DESCRIPTION OF THE INVENTION

In the wash compositions of the present invention, acyl isethionates are anionic surfactants suitable for use. These include alkoxylated isethionates such as those described in llardi et al., U.S. Pat. No. 5,393,466, entitled “Fatty Acid Esters of Polyalkoxylated isethionic acid. The isethionate surfactants can include the reaction product of fatty acids esterified with isethionic acid and neutralized with a base like, for example, sodium or potassium hydroxide. The acyl isethionate surfactant can have the general formula:

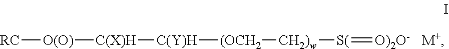

where R is a C₅ to C₁₉ saturated or unsaturated alkyl, w is an integer from 0 to 4 (1 to 4 for the alkoxylated option), X and Y are each independently hydrogen or an alkyl group having 1 to 4 carbons and M is a counter ion such as a Na⁺, K⁺, NH₄⁺, mixture thereof or the like.

It is also within the scope of the invention, when w is 0, for either the carbon alpha or beta to the sulfonate group to have one C₁₋₄ alkyl substitution in place of hydrogen, preferably a C₁₋₃ alkyl substitution and most preferably, a methyl group. It is further within the scope of the invention for R to have a degree of unsaturation, and typically, no more than 2, and preferably, no more than 1 double bond.

Illustrative examples of the isethionates suitable for use in the wash composition include sodium capryl isethionate, sodium caproylyl isethionate, sodium capryl methyl isethionate, sodium caproylyl methyl isethionate, sodium cocoyl isethionate, sodium cocoyl methyl isethionate, sodium lauroyl isethionate, sodium lauroyl methyl isethionate, potassium lauroyl isethionate, potassium lauroyl methyl isethionate, sodium oleoyl isethionate, sodium oleoyl methyl isethionate, sodium stearoyl isethionate, sodium stearoyl methyl isethionate, sodium myristoyl isethionate, sodium myristoyl methyl isethionate, sodium palmitoyl isethionate, sodium palmitoyl methyl isethionate, ammonium cocoyl isethionate, ammonium cocoyl methyl isethionate, or a mixture thereof. In an embodiment of the invention, isethionate selected for use comprises sodium lauroyl isethionate, sodium cocoyl isethionate or a mixture thereof. In another embodiment, the isethionate selected consists essentially of or consists of sodium lauroyl isethionate, sodium cocoyl isethionate or a mixture thereof. In an often-desired embodiment, the isethionate used is sodium lauroyl isethionate or sodium cocoyl isethionate.

Additional anionic surfactants suitable for use in the wash compositions of the present invention include acyl taurates. Illustrative examples include sodium methyl lauroyl taurate, potassium methyl lauroyl taurate, sodium methyl myristoyl taurate, potassium methyl myristoyl taurate, ammonium methyl myristoyl taurate, sodium methyl cocoyl taurate, potassium methyl cocoyl taurate, ammonium methyl cocoyl taurate, sodium methyl oleoyl taurate, potassium methyl oleoyl taurate, ammonium methyl oleoyl taurate, sodium lauroyl taurate, potassium lauroyl taurate, ammonium myristoyl taurate, sodium cocoyl taurate, potassium oleoyl taurate, mixtures thereof or the like. In an embodiment of the invention, the taurate used is sodium methyl lauroyl taurate.

Even other anionic surfactants suitable for use include acyl glycinates and salts thereof. Such glycinates include C₈ to C₂₀, and preferably, C₁₀ to C₁₈ and most preferably, C₁₂ to C₁₆ or C₁₂ to C₁₄ acyl glycinates. Illustrative and nonlimiting examples of the glycinates that may be included are sodium lauroyl glycinate, sodium myristoyl glycinate, sodium cocoyl glycinate, potassium lauroyl glycinate, sodium myristoyl glycinate, potassium cocoyl glycinate or a mixture thereof. In an embodiment of the invention, sodium cocoyl glycinate, potassium cocoyl glycinate or a mixture thereof are often preferred when acyl glycinates are used. In another embodiment, sodium or potassium lauroyl glycinate or both may also be used in the wash composition of the invention. In even another embodiment of the invention, the acyl portion of the glycinates that may be selected for use is preferably saturated but suitable to be unsaturated with no more than 2 double bonds, including conjugated double bonds.

Acyl glutamates (and salts thereof) may also optionally be included as anionic surfactant in the wash composition. Glutamates such as C₈ to C₂₀, and preferably, C₁₀ to C₁₈ and most preferably, C₁₂ to C₁₆ or C₁₂ to C₁₄ acyl glutamates where the acyl portion is preferably saturated are suitable for optional use. As is the case with glycinates, the hydrophobic portion of the glutamates may be unsaturated with no more than 2 double bonds, including conjugated double bonds. Illustrative yet nonlimiting examples of the glutamates that may be used include sodium capryloyl glutamate, sodium lauroyl glutamate, sodium myristoyl glutamate, sodium cocoyl glutamate, sodium stearoyl glutamate, dipotassium capryloyl glutamate, dipotassium undecylenoyl glutamate, disodium capryloyl glutamate, disodium cocoyl glutamate, disodium lauroyl glutamate, disodium stearoyl glutamate, disodium undecylenoyl glutamate, potassium capryloyl glutamate, potassium cocoyl glutamate, potassium lauroyl glutamate, potassium myristoyl glutamate, potassium stearoyl glutamate, potassium undecylenoyl glutamate, sodium olivoyl glutamate, sodium palmitoyl glutamate, sodium undecylenoyl glutamate, disodium cocoyl glutamate or a mixture thereof. In an embodiment of the invention, sodium lauroyl glutamate, sodium cocoyl glutamate, sodium stearoyl glutamate or a mixture thereof is preferred when glutamates are desired for optional use. In another embodiment, if a glutamate is used, sodium myristoyl and/or sodium cocoyl glutamate may often be desired.

Still other anionic surfactants suitable to be selected for use include alkyl sulfates, alkyl sulfonates, alkyl sulfosuccinates, alkyl ether sulfosuccinates, acyl sarcosinates or a mixture thereof (including any salts thereof).

As noted herein, the wash composition is, preferably, substantially free of surfactants that are sulfated (e.g., sulfate-based or comprising surfactants, e.g., sodium lauryl sulfate) and including those that are alkoxylated (e.g., sodium lauryl ether sulfate)). In an embodiment of the invention sulfated surfactants may be used, and again, preferably, at an amount of less than 1% by weight of the wash composition. If optionally included, the alkyl sulfates can include C₈-C₂₀ alkyl sulfates and/or alkoxylated versions such as sodium lauryl ether sulfate, sodium pareth ether sulfate or a mixture thereof where the ethoxylated portion (EO) is typically on average 1-3 EO groups. When selected, sodium lauryl sulfate, sodium lauryl ether sulfate, sodium pareth sulfate, ammonium lauryl sulfate or a mixture thereof are illustrative examples that may be included as an option for use.

As to additional sulfonates and their salts that may be selected for use as anionic surfactants, the same can include alkyl sulfonates, alkyl glyceryl ether sulfonates or a mixture thereof where the alkyl portion is typically a C₈₋₁₈ or C₁₀₋₁₆ hydrocarbon.

Other anionic surfactants suitable for use include succinates (including their salts) like disodium oleamido MIPA sulfosuccinate, disodium oleamido MEA sulfosuccinate, disodium lauryl sulfosuccinate, disodium laureth sulfosuccinate, diammonium lauryl sulfosuccinate, diammonium laureth sulfosuccinate, dioctyl sodium sulfosuccinate, disodium oleamide MEA sulfosuccinate, sodium dialkyl sulfosuccinate, or a mixture thereof. For the avoidance of doubt, MIPA and MEA refer to monoisopropanolamine and monoethanolamine, respectively.

Acyl sarcosinates suitable for use as anionic surfactant include those having a C₈-C₂₀ or C₁₀-C₁₈ or C₁₂-C₁₈ acyl group. Illustrative examples of the sarcosinates that may optionally be used include sodium lauroyl sarcosinate, sodium cocoyl sarcosinate, sodium stearoyl

In an embodiment of the invention, at least one anionic surfactant included is sodium lauryl sulfosuccinate, sodium myristoyl sulfosuccinate, sodium cocoyl sulfosuccinate, sodium stearoyl sulfosuccinate, sodium laureth sulfosuccinate, sodium pareth sulfosuccinate, disodium laureth sulfosuccinate, disodium lauryl sulfosuccinate, diethylhexyl sodium sulfosuccinate or a mixture thereof. In one embodiment of the invention, acyl sarcosinate makes up from 0.5 to 10% or from 1 to 8% or from 3 to 6% or 4 to 5% by weight of the total weight of anionic surfactant in the wash composition with sodium cocoyl sarcosinate, sodium lauroyl sarcosinate or a mixture thereof often being preferred if a sarcosinate is included.

In yet another embodiment, anionic surfactant may include sodium methyl 2-sulfolaurate or disodium 2-sulfolaurate or both.

In a preferred embodiment of the invention, anionic surfactant included is sodium lauroyl isethionate, sodium cocoyl isethionate or a mixture a mixture thereof.

In still another embodiment of the invention, anionic surfactant suitable for use includes fatty acid amides of an amino acid, i.e., acyl alaninates. Examples suitable for use include triethanolamine cocoylalaninate, sodium cocoylalaninate, triethanolamine lauroylalaninate, sodium lauroylalaninate or a mixture thereof.

Still other anionic surfactants suitable to include in the wash compositions of the present invention are alpha olefin sulfonates represented by the structure:

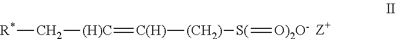

where R* is a C₆ to C₁₆ linear, branched, saturated or unsaturated hydrocarbon. When unsaturated R* preferably has 1 or 2, and most preferably, 1 double bond (i.e., each carbon atom of the bond being sp² hybridized). In an embodiment of the invention, R* is C₆ to C₁₆, and preferably, C₆ to C₁₄, and most preferably, C₈ to C₁₄, and even more preferably, C₁₀ to C₁₂. In an embodiment of the invention, at least 65%, and preferably at least 75%, and most preferably, from 80 to 100% or from 90 to 100% or from 95 to 100% or from 82 to 94% or from 82 to 90% or from 78 to 87% by weight of the alpha olefin sulfonate used is one where R* is C₁₀ to C₁₂ based on total weight of alpha olefin sulfonate in the wash composition. In even another embodiment, from 96 to 100% or from 92 to 99% or from 90 to 96% by weight of the alpha olefin sulfonate used is one where R* is C₁₀ to C₁₂ based on total weight of alpha olefin sulfonate in the wash composition.

Z⁺ is a counter ion that typically includes sodium, ammonium and/or potassium ions or a mixture thereof.

In yet another embodiment, no more than 12%, and preferably, no more than 7%, and most preferably, no more than 5% by weight of the total weight of surfactant used is alpha olefin sulfonate. In still another embodiment, from 0.0001 to 4% or 0.0% by weight of the total weight of surfactant is alpha olefin sulfonate.

As to the amphoteric surfactant suitable for use in the wash composition of the invention, the same can include betaines like lauryl betaine, lauryldimethyl betaine, coco betaine, cocamidopropyl betaine, laurylamidopropyl betaine, a mixture thereof or the like.

Still other amphoteric surfactants suitable for use include lauryl hydroxysultaine, cocamidopropyl hydroxysultaine or mixtures thereof. Even other amphoteric surfactants that may be used in the wash composition include alkyl amidopropyl hydroxysultaines including palmityl, stearyl and/or oleyl amidopropyl hydroxysultaine, and most preferably, palmityl amidopropyl hydroxysultaine. Other amphoteric surfactants suitable for use include behenyl betaine, capryl/capramidopropyl betaine, stearyl betaine, myristyl hydroxysultaine or a mixture thereof. In an embodiment of the invention, the amphoteric surfactant used comprises cocamidopropyl betaine, lauryl sulfobetaine, cocodimethyl carboxymethyl betaine, laurylhydroxy sulfobetaine, cocoamidopropylhydroxyl sulfobetaine, lauryl hydroxysultaine, a mixture thereof or the like. Additional amphoteric surfactants that may be used include C₁₀₋₁₈ alkylamidopropylamine N-oxides like laurylamidopropylamine N-oxide, C₁₀₋₁₈ alkyldimethylamine N-oxides like lauryldimethylamine N-oxide, C₁₀₋₁₈ alkylbetaines like laurylbetaine, or mixtures thereof.

In still another embodiment, the amphoteric surfactant used comprises lauryl sulfobetaine, lauryl hydroxysultaine or a mixture thereof.

Still other surfactants suitable for use having charge impacted by pH include cocamine oxide, lauramine oxide, myristamine oxide, palmitamine oxide, stearamine oxide, oleamine oxide, cocamidopropyl amine oxide, lauryl amidopropyl amine oxide, myristyl amidopropyl amine oxide, palmityl amidopropyl amine oxide, stearyl amidopropyl amine oxide, oleamidopropyl amine oxide or a mixture thereof. Additional optional examples of the amphoteric surfactants suitable for use include sodium lauroamphoacetate, sodium cocoamphoacetate, sodium lauroamphoacetate, sodium cocoamphoacetate, cocamphodipropionate or a mixture thereof.

In an embodiment of the invention the amphoteric selected can be at least 80%, and preferably, at least 85%, and most preferably, at least 90% (or 90 to 100% or 94 to 100% or 94 to 98% or 100%) by weight amine oxide whereby such amine oxide can have the formula: R^{x}—N⁺(R^{y})₂—O— where R^{x} is a C₈₋₂₀ alkyl, and preferably, a C₁₀₋₁₈ alkyl, and most preferably, a C₁₂₋₁₈ alkyl (or C₁₂₋₁₆ alkyl) and R^{y} is H or a C₁₋₆ alkyl or C₁₋₄ alkyl or C₁₋₃ alkyl or C₁₋₂ alkyl or —CH₃. In another embodiment of the invention, amine oxide suitable for optional use is cocoyl amine oxide, lauramine oxide, myristamine oxide, palmitamine oxide, stearamine oxide, oleamine oxide or a mixture thereof.

In yet another embodiment, the amphoteric surfactant used is from 88 to 100% or from 92 to 100% or from 93 to 98% or from 95 to 100% by weight cocamidopropyl hydroxysultaine, cocamidopropyl betaine or a mixture thereof based on total weight of amphoteric surfactant in the wash composition. In still another embodiment, the amphoteric surfactant used is 95 to 100% by weight cocamidopropyl betaine or 100% by weight cocamidopropyl betaine.

As noted herein, the amphoteric surfactant comprises 25% or less, and preferably, 18% or less, and most preferably, 10% or less, or from 0.0 to 8%, or from 0.01 to 4%, or 0.0% by weight hydrophobic portion derived from triglyceride based on total weight of hydrophobic portion present on the amphoteric surfactant. In another embodiment, the amphoteric surfactant used comprises from 0.001 to 0.3% by weight hydrophobic portion derived from triglyceride based on total weight of hydrophobic portion present on the amphoteric surfactant, and preferably, no, 0.0% by weight, hydrophobic portion derived from triglyceride based on total weight of hydrophobic portion present on the amphoteric surfactant.

Anionic surfactant typically makes up from 2.5 to 8% or from 2.5 to 5.5% or from 2.75 to 5% or from 3 to 5% or from 3.5 to 5% or from 3.7 to 4.7% by weight of the wash composition, with the proviso that if the anionic surfactant comprises acyl isethionate, the total weight of acyl isethionate included does not exceed 3.6% by weight of the total weight of the wash composition. In an embodiment of the invention, if used, acyl isethionate makes up from 0.01 to 3.6%, or from 0.5 to 3.5%, or from 0.8 to 3.3%, or from 1 to 3.2%, or from 1 to 3.2%, or from 2 to 3.2%, or from 1 to 2%, or from 1 to 3.1, or from 2 to 3.1% by weight of the wash composition. In another embodiment, no acyl isethionate (0.0% by weight) is included in the wash composition.

Amphoteric surfactant often makes up from 4 to 11%, and preferably, from 4.5 to 10%, and most preferably, from 5 to 10% or from 5.5 to 9.5% or from 6 to 9% or from 6.5 to 8.5% or from 6.7 to 8.2% or from 7.3 to 8.3% by weight of the wash composition.

In an embodiment of the invention, amphoteric surfactant makes up from 50 to 90%, or from 52 to 75% or from 55 to 70% or from 58 to 70% or from 60 to 70% or from 62 to 67% by weight of the total weight of anionic surfactant and amphoteric surfactant in the wash composition.

The wash composition comprises from 4 to 18% by weight total surfactant. In still another embodiment of the invention, the wash composition comprises from 5 to 17%, or from 6 to 16%, or from 7 to 15.5%, or from 8 to 15%, or from 9 to 14.5%, or from 10 to 13.5%, or from 11 to 13% by weight of the wash composition.

In another embodiment of the invention, additional surfactants suitable for optional use include imidazolines, sodium acyl amphoacetates, sodium acyl amphopropionates, disodium acyl amphodiacetates and disodium acyl amphodipropionates where the acyl (i.e., alkanoyl group) can comprise a C_{7-C18} alkyl portion and the same may make up from 0.001 to 0.8% or from 0.1 to 0.6% or from 0.1 to 0.3% by weight of the wash composition.

The wash composition of the present invention can optionally comprise from 0.28 to 1.5%, and preferably, from 0.3 to 1.2%, and most preferably, from 0.3 to 1% or from 0.35 to 0.9% or from 0.38 to 0.8% or from 0.4 to 0.7% or from 0.4 to 0.5% by weight of nonionic surfactant.

Nonionic surfactant which may be included can be the reaction products of compounds having a hydrophobic group and a reactive hydrogen atom, for example aliphatic alcohols, acids, amides or alkylphenols with alkylene oxides, especially ethylene oxide either alone or with propylene oxide. Specific nonionic surfactant compounds are alkyl (C₆-C₂₂) phenols, ethylene oxide condensates, the condensation products of aliphatic (C₈-C₁₈) primary or secondary linear or branched alcohols with ethylene oxide, and products made by condensation of ethylene oxide with the reaction products of propylene oxide and ethylenediamine.

In an embodiment of the invention, nonionic surfactants selected can include fatty alcohol ethoxylates like Steareth-2, Steareth-10, Ceteth-20, Oleth-20 Beheneth-20 or a mixture thereof. In another embodiment, fatty acid ethoxylates may be used and they include lauric acid ethoxylate, coconut fatty acid ethoxylate, myristic acid ethoxylate, palmitic acid ethoxylate, stearic acid ethoxylate, oleic acid ethoxylate, soya bean fatty acid ethoxylate or a mixture thereof.

The nonionic may also include a sugar amide, such as a polysaccharide amide. Specifically, the surfactant may be one of the lactobionamides described in U.S. Pat. No. 5,389,279 to Au et al., entitled “Compositions Comprising Nonionic Glycolipid Surfactants or it may be one of the sugar amides described in U.S. Pat. No. 5,009,814 to Kelkenberg, entitled “Use of N-Poly Hydroxyalkyl Fatty Acid Amides as Thickening Agents for Liquid Aqueous Surfactant Systems”.

In an embodiment of the invention and if used, the nonionic surfactant may comprise an alkyl polyglucoside represented as:

R¹—O—(S)ₙ, where R¹ is a C₆ to C₂₀ or C₈ to C₁₈ or C₁₀ to C₁₆ linear or branched alkyl and S is a saccharide group of 5 or 6 carbons such as glucose, xylose, lactose, fructose and/or mannose, and preferably, is glucose, and n represents the degree of polymerization and may have a value of from 1 to 12 or from 1 to 10, and most preferably, from 1.2 to 1.6 or from 1.3 to 1.5.

In yet another embodiment of the invention, the nonionic surfactant when used includes polysorbate (like polysorbate 20), cocamide monoethanolamine (cocamide MEA) or a mixture thereof. In even another embodiment, the nonionic surfactant when used is cocamide MEA. In still another embodiment of the invention, cocamide MEA makes up from 0.2 to 0.5%, by weight of the wash composition.

Fatty acids like C₁₀ to C₂₀ or C₁₂ to C₁₈ or C₁₆ to C₁₈ fatty acids and amide and alcohol derivatives thereof (and any mixtures of the same) may be included in the wash composition of the invention. In an embodiment of the invention and when used, such components typically make up from 0.01 to 1%, or from 0.05 to 0.8%, or from 0.06 to 0.5%, or from 0.09 to 0.45%, or from 0.09 to 0.4%, or from 0.09 to 0.2% by weight of the wash composition. Preferred for use are fatty acids including lauric acid, palmitic acid, stearic acid, or a mixture thereof. In another embodiment of the invention, both lauric acid and stearic acid are used at a weight ratio, respectively, from 1:1 to 1:4 or from 1:2 to 1:3.5 or from 1:2 to 1:3 or from 1:1. In yet another embodiment, the fatty acid used is at least 50% by weight, and preferably, from 55 to 90%, and most preferably, from 60 to 80% or from 60 to 83% or from 65 to 75% by weight stearic acid.

Cationic surfactants may optionally be used, and these include cetyltrimethylammonium bromide, cetyltrimethylammonium chloride, cetylpyridium chloride, behentrimonium chloride, stearalkonium chloride, lauryltrimethylammonium chloride, mixtures thereof or the like. If optionally used, such surfactants will often make up from 0.01 to less than 1%, or from 0.1 to 0.7% or from 0.2 to 0.5% by weight of the wash composition. In an embodiment of the invention, no (0.0%) cationic surfactant is used.

Inorganic salt is an optional ingredient to aid in composition consistency and water activity adjustment. Typical salts may be used like NaCl, KCl, MgCl₂, CaCl₂, mixtures thereof or the like. If used, such salt makes up less than 1.65%, and preferably, less than 1.6% and most preferably, from 0.05 to 1.55% or from 0.5 to 1.5%, or from 1 to 1.5% or from 1.1 to 1.45% by weight of the wash composition. In an embodiment of the invention the wash composition is less than 0.6% by weight salt or less than 0.5% by weight salt or from 0.001 to 0.5% by weight salt or 0.0% by weight salt.

Fatty acid sources for the surfactants described herein may, for example, include vegetable, soy, nut, jojoba, avocado, olive, seed oil (e.g., sunflower, linseed, rapeseed, moringa), fruit (e.g., mango, papaya, red berries, coconut), petrolatum, palm kernel and/or palm oil. In an embodiment of the invention, less than 5%, and preferably, less than 2%, and most preferably, less than 1% (or 0.0%) palm kernel oil is used to make surfactant included in the present invention. In another embodiment of the invention, from 0.001 to 70%, and preferably, from 0.01 to 60%, and most preferably, from 1 to 50% (or 2 to 39%) by weight of total surfactant used in the present invention is derived from palm oil and/or palm kernel oil. In even another embodiment, surfactant in the present invention is less than 5% by weight (or 0.0% by weight) palm kernel oil derived.

In a further embodiment of the invention, from 0.0 to 15% or from 0.01 to 12% or from 0.5 to 10% by weight of any of the surfactants used in the present invention may have hydrophobic portion with carbon recovered from purple carbon, and that is, carbon recovered from carbon dioxide or carbon monoxide or methane waste gas (or mixtures thereof) via biotechnology that utilizes microbial gas fermentation. It is also suitable in the invention to utilize carbon sources recovered from algal oils, microbial enhanced oil recovery processes and from plants like the oil recovered from Macauba pulp oil (Acrocomia aculeata).

As to the peroxisome proliferator-activated receptor (PPAR agonist) suitable to use in the wash composition of the present invention, the wash composition will comprise more than 0.1 to 2%, and preferably, from 0.15 to 1%, and most preferably, from 0.15 to 0.7% or from 0.18 to 0.5% or from 0.2 to 0.35% or from 0.2 to 0.3% or from 0.2 to 0.28% by weight of the PPAR agonist where such agonist is at least 65%, and preferably, at least 75%, and most preferably, from 80 to 100% or from 85 to 100% or from 90 to 100% or from 60 to 90% or from 90 to 98% or 100% by weight 12-hydroxystearic acid based on total weight of the PPAR agonist in the wash composition. Other PPAR agonists suitable for use with 12-hydroxystearic acid are preferably PPAR-alpha agonists including but not limited to 10-hydroxystearic acid, petroselinic acid, conjugated linoleic acid, 12-hydroxy-9-cis-octadecanoic acid (ricinoleic acid), palmitoleic acid, stearolic acid or a mixture thereof.

Polyols (i.e., humectants) are suitable for use in the wash compositions of the present invention whereby the same are limited only to the extent that they are suitable for use in a consumer product. Illustrative and nonlimiting examples of the polyols that may be used include sorbitol, glycerol (glycerin), mannitol, xylitol, maltitol or mixtures thereof. In an embodiment of the invention, the polyol used is at least 50% by weight glycerol, based on total weight of the polyol used. In another embodiment of the invention, the polyol used is all glycerol (100% by weight). Polyol, when used, will typically make up from 0.5 to 17%, or from 5 to 7% by weight of the wash composition, and preferably, from 0.5 to 6% by weight of the wash composition, and most preferably, from 0.5 to 4% (or from 0.7 to 3% or 0.7 to 2.5% or from 0.8 to 1.5%) by weight of the wash composition. In an embodiment of the invention, no (0.0% by weight) humectant is used in the wash composition.

Water typically makes up from 70 to 92%, and preferably, from 72 to 90%, and most preferably, from 75 to 88% or from 78 to 87% by weight of the wash composition.

Adjusters suitable to modify/buffer the pH may optionally be used. Such pH adjusters include triethylamine, NaOH, KOH, H₂SO₄, HCl, C₆H₈O₇ (i.e., citric acid) or mixtures thereof. The pH adjusters are added at amounts to yield the desired final pH (as defined from 5.65 to 8.5). The pH values may be assessed with commercial instrumentation such as a pH meter made commercially available from Thermo Scientific®. Adjuster, when used, is typically present at from 0.0001 to 0.7% or from 0.0002 to 0.3% or from 0.02 to 0.22% or from 0.03 to 0.08% by weight of the wash composition.

In an embodiment of the invention, the wash composition may optionally comprise polymeric suspending agent to contribute to, for example, product homogeneity, consumer skin appearance and sensory experience. Polymeric suspending agent, if used, typically makes up less than 0.6% by weight, and preferably, less than 0.4%, and most preferably, less than 0.3%, (or less than 0.22% or less than 0.1% or less than 0.05%) by weight of the wash composition of the invention.

Illustrative examples of optional suspending agent suitable for use include those which are a fermentation derived cellulose like Arbalon R-50® and Arbalon R-50® EV made available from Lubrizol. Such products comprise glycerin, cellulose and cellulose gum with 1.3 to 2.5 or 1.5 to 2.35 or 1.55 to 2.25 times more cellulose than cellulose gum. Glycerin typically makes up from 2.45 to 3.6 or from 2.55 to 3.5 or from 2.75 to 3.3 times more than the total amount of cellulose and cellulose gum. Often, the desired cellulose is one derived from a microbial fermentation process.

The wash composition may optionally comprise thickening agent to additionally contribute to viscosity and sensory attributes of the composition. The same, if optionally used, typically makes up from 0.001 to 1.5% by weight, and preferably, from 0.06 to 0.4%, and most preferably, less than 0.3%, (or from 0.07 to 0.5% or less than 0.25% or less than 0.15% or less than 0.1%) by weight of the wash composition.

Illustrative examples of the optional thickening agent suitable for use include hydroxyethyl cellulose, hydroxypropyl methylcellulose, acrylates/steareth-20 methacrylate copolymer, PEG 120 methyl glucose trioleate (and) propanediol, acrylates/beheneth 25 methacrylate copolymer, xanthan gum, carbomer, acrylates/C₁₀₋₃₀ alkyl acrylate crosspolymer, guar hydroxypropytrimonium chloride, carrageenan, xanthan gum/locust bean gum, xanthan gum/guar gum, cellulose gum, microcrystalline cellulose, cellulose gel, hydroxyethyl cellulose, hydroxypropyl cellulose, sodium carboxymethylcellulose, hydroxymethyl or carboxymethyl cellulose, methyl cellulose, ethyl cellulose, guar gum, gum karaya, gum tragacanth, gum Arabic, gum acacia, gum agar, xanthan gum, Versathix™ (PEG-150 Pentaerythrityl Tetrastearate (and) PPG-2 Hydroxyethyl cocamide and water), Aristoflex AVC (ammonium acryloyldimethyltaurate/VP copolymer), Carbopol® Ultrez 10 and 20 (crosslinked polyacrylic acid); alkaline soluble emulsion polymers such as Aculyn 28, Acuyln 22 or Carbopol Aqua SF1; cationic polymer such as modified polysaccharides including cationic guar available from Aventis under the trade name Jaguar C13S, Jaguar C14S, Jaguar C17, or Jaguar C16; cationic modified cellulose such as UCARE Polymer JR 30 or JR 40 from Amerchol, N-Hance 3000, N-Hance 3196, N-Hance GPX 215 or N-Hance GPX 196 from Ashland, Inc., synthetic cationic polymers such as MerQuat 100, MerQuat 280, Merquat 281 and Merquat 550 by Nalco, cationic starches, e.g., StaLok® 100, 200, 300 and 400 made by Galactasol 800 series by Henkel, Inc., Quadrosoft Um-200, Polyquaternium-24, Structure® ZEA (a hydroxypropyl modified corn starch) or Structure® XL, a cross-linked pregelatinized hydroxypropyl starch phosphate or a mixture thereof.

Even other optional suitable thickeners that may be used include cationic polymers such as those which are salts of hydroxyethyl cellulose reacted with a trimethyl ammonium substituted epoxide like Polyquaternium-10, made commercially available by Dow® as UCARE™ Polymer JR-125, UCARE Polymer JR-400, UCARE Polymer KF, UCARE Polymer JR-30M, UCARE Polymer LR-400, UCARE Polymer LR-30M, UCARE Polymer LK mixtures thereof or the like.

Other suitable cationic polymers that can be selected for use include those known as hydrophobically-modified cationic conditioning polymers such as those made commercially available by Dow® under the names SoftCAT™ SL 5, SoftCAT SL 30, SoftCAT SL 60, SoftCAT SL 100, SoftCAT SK-L, SoftCAT SK-M, and SoftCAT SK-H.

As noted, in an embodiment of the invention the wash composition is substantially free of acrylate-based thickeners.

Oil may optionally be included in the wash composition of this invention. If used, oil may make up from 0.001 to 0.35% or from 0.005 to 0.32% or from 0.008 to 0.28% by weight of the wash composition. Oils that may be included are any of those suitable to contact skin, and preferably, those that are natural and sustainable. Illustrative examples of such oils include silicone oils and/or mineral oil, but those that are naturally sourced and sustainable like, arachis oil, castor oil, coconut oil, corn oil, cotton seed oil, olive oil, rapeseed oil, safflower seed oil, sesame seed oil, soybean oil, avocado oil, macadamia nut oil, argan oil, pomegranate oil, argan Moroccan oil, blueberry oil, raspberry oil, walnut oil, pecan oil, peanut oil, bayberry oil, mango seed oil, jojoba oil, hydrolyzed jojoba oil, moringa oil, coffee seed oil, mixtures thereof or the like are typically desired. If a silicone oil is optionally used, the same can include, for example, PEG-3 Dimethicone, PEG-8 Dimethicone, PEG-9 Dimethicone, PEG-10 Dimethicone, PEG-11 Methyl ether dimethicone, PEG-12 Dimethicone, PEG-14 Dimethicone, PEG-17 Dimethicone, PEG-32 Dimethicone mixtures thereof or the like. As noted, however, the wash composition of the present invention is preferably substantially free of silicone oil. In an embodiment of the invention, from 0.01 to 0.32, or from 0.1 to 0.29, or from 0.2 to 0.28% by weight moringa oil is included in the wash composition.

The oil used can have a droplet size from 20 nm to 10 microns or from 50 nm to 2 microns or from 30 nm to 1 micron. In an embodiment of the invention, oil is provided as a nanoemulsion with droplet sizes ranging from 50 to 800 nm or from 60 to 700 nm from 125 to 500 nm. Optional water-soluble cosmetic agents suitable for use in the wash composition of the invention are limited only to the extent that they are capable of being used in a consumer product at the desired pH described.

Illustrative examples of the skin benefit agents suitable to include in the wash composition are acids, like amino acids, such as arginine, valine or histidine. Additional water-soluble benefit agents suitable for use include vitamin B2, niacinamide (vitamin B3), vitamin B5 (Panthenol), vitamin B6, folic acid (vitamin B9), vitamin C, mixtures thereof or the like. Water soluble derivatives of such vitamins may also be employed. For instance, vitamin C derivatives such as ascorbyl tetraisopalmitate, magnesium ascorbyl phosphate and ascorbyl glycoside may be used alone or in combination with each other. Other water-soluble benefit agents suitable for use include 4-ethyl resorcinol, water soluble extracts of sage, aloe vera, green tea, grapeseed, thyme, chamomile, yarrow, cucumber, liquorice, rosemary extract or mixtures thereof. Water soluble sunscreens like ensulizole (phenylbenzimidizole sulfonic acid), disodium phenyl dibenzimidazole tetra sulfonate or mixtures thereof may also be included. Total amount of optional water-soluble benefit agents (including mixtures) when present in the wash composition may range from 0.001 to 10%, preferably from 0.001 to 5%, and most preferably, from 0.01 to 8% (or from 1 to 6% or from 1 to 3%) by weight of the wash composition.

It is also within the scope of the present invention to optionally include oil soluble cosmetic agents (i.e., non-water-soluble agents defined as less than 1.0 g of such agent (solute) can be dissolved in 100 ml of 25° C. water (solvent) at atmospheric pressure). The only limitation with respect to such oil soluble cosmetic agents are that the same are suitable for use in a consumer product.

Illustrative examples of the types of oil soluble cosmetic agents that may optionally be used in the wash composition of the invention include components like sunscreens and including but not limited to ethylhexylmethoxycinnamate, bis-ethyl hexyloxyphenol methoxyphenol triazine, 2-ethylhexyl-2-cyano-3,3-diphenyl-2-propanoic acid, drometrizole trisiloxane, 3,3,5-trimethyl cyclohexyl 2-hydroxybenzoate, 2-ethylhexyl-2hydroxybenzoate or a mixture thereof.

Other optional oil soluble cosmetic agents suitable for use include resorcinols like thiamidol (isobutylene thiazolyl resorcinol), 4-hexyl resorcinol, 4-phenylethyl resorcinol, 4-cyclopentyl resorcinol, 4-cyclohexyl resorcinol 4-isopropyl resorcinol or a mixture thereof. Also, 5-substituted resorcinols like 4-cyclohexyl-5-methylbenzene-1,3-diol, 4-isopropyl-5-methylbenzene-1,3-diol, mixtures thereof or the like may be used. The 5-substituted resorcinols, and their synthesis are described in commonly assigned U.S. Published Patent Application No. 2016/0000669A1.

Even other oil soluble cosmetic agents suitable for use include omega-3 fatty acids, omega-6 fatty acids, climbazole, farnesol, ursolic acid, myristic acid, geranyl geraniol, oleyl betaine, cocoyl hydroxyethyl imidazoline, vitamin E (and its derivatives including alpha-tocopherol acetate), hexanoyl sphingosine, terpineol, thymol, mixtures thereof or the like.

In an embodiment of the invention, the optional oil soluble cosmetic agent used is a retinoic acid precursor. In one embodiment of the invention, the retinoic acid precursor is retinol, retinal, retinyl propionate, retinyl palmitate, retinyl acetate or a mixture thereof. Retinyl propionate, retinyl palmitate and mixtures thereof are typically preferred when used.

When optional oil soluble cosmetic agent is used in the wash composition, such cosmetic agent typically makes up to 2% (or to 1.5%), and preferably, from 0.001 to 1.2%, and most preferably, from 0.05 to 0.85% by weight of the wash composition with the proviso that such weight percents exclude the amount of PPAR agonist present in the wash composition.

Conventional preservatives can desirably be incorporated into the wash composition to protect against the growth of potentially harmful microorganisms. Cosmetic chemists are familiar with appropriate preservatives and routinely choose them to satisfy the preservative challenge test and to provide product stability. Suitable traditional preservatives that may be used include those traditionally found in consumer products. Preservatives suitable for preferred use are iodopropynyl butyl carbamate, phenoxyethanol, hydroxyacetophenone, ethylhexylglycerine, imidazolidinyl urea, sodium dehydroacetate, benzyl alcohol, sodium benzoate and mixtures thereof. Other preservatives suitable for use include sodium dehydroacetate, chlorophenesin and decylene glycol. The preservatives should be selected having regard for the use of the composition and possible incompatibilities between the preservatives and other ingredients included. Preservatives are preferably employed in amounts ranging from 0.01% to 2.3% (or 0.1 to 2% or 0.1 to 1.5%) by weight of the total weight of the wash composition. Also preferred is a preservative system with hydroxyacetophenone alone or in a mixture with other preservatives.

While suitable for use, but in an embodiment of the invention, the wash composition is substantially free of hydantoins, parabens and isothiazolinones.

In another embodiment, the preservative used is phenoxyethanol, iodopropynyl butyl carbamate or a mixture thereof (present at amounts from 0.2 to 1% or 0.3 to 0.9% or 0.4 to 0.8% by weight of the wash composition). In even another embodiment, 1,2-octanediol is used with preservative like sodium benzoate, phenoxyethanol, iodopropynyl butyl carbamate or a mixture of the same where the vicinal diol, if used, makes up from 0.5 to 1.5% or from 0.6 to 1.3% or from 0.7 to 1.2% times the amount of preservative used. Such diols are common emollients. Lipidated glycines are also suitable for use to boost preservative efficacy. If selected for use, such lipidated component may make up from 0.01 to 0.7% or from 0.1 to 0.6% or from 0.15 to 0.4% by weight of the wash composition. Often preferred lipidated components suitable for use are capryloyl glycine, undecylenoyl or a mixture thereof. In an embodiment of the invention, no (0.0% by weight) vicinal diol and lipidated glycines are used.

In even another embodiment, the wash composition made according to the invention may comprise thymol, terpineol, chloroxylenol, zinc pyrithione, piroctone olamine or a mixture thereof. In still another embodiment, the wash compositions may comprise N-acetyl methionine, N-acetyl cysteine (D, L isomers) or both. When selected for optional use, the same may make up alone or collectively from 0.01 to 3% or from 0.1 to 2% or from 0.2 to 1.5% or from 0.1 to 1% or from 0.1 to 0.6% by weight of the wash composition.

Fragrances, fixatives, chelators (like EDTA, citric acid, salicylic acid, sodium gluconate, triethanolamine), colorants (like caramel, beet juice, beta-carotene, vegetable juice or mixtures of the same), hydrated silica, alcohols (like polypropylene glycol, PPG-7), and exfoliants may optionally be included in the wash composition. Each of these substances may range from about 0.03 to about 4%, preferably between 0.01 and 3% (or from 0.05 to 1.2% or 0.07 to 1%) by weight of the total weight of the wash composition. To the extent exfoliants are used, those selected should be of small enough particle size so that they do not impede the performance of any packaging used to dispense the wash composition of the invention. In an embodiment of the invention, less than 0.7% or less than 0.4% or from 0.1 to 0.3% or 0.0% by weight EDTA is present in the composition.

In another embodiment of the invention, the wash composition will be substantially free of a sulfate-based surfactant and comprise at least one of botanical microspheres, thiamidol, oleyl adapalenate, hyaluronic acid, 10-hydroxystearic acid, 12-hydroxystearic acid, mandelic acid, lactic acid, caffeine, tretinoin ((all-E)-3,7-dimethyl-9-(2,6,6-trimethyl-1 cyclonexen-1-yl)-2,4,6,8-nonatetraenoic acid), epigallocatechin gallate, adapalene (6-[3-(1-adamantyl)-4-methoxyphenyl]naphthalene-2-carboxylic acid), 2-mecaptonicotinoyl glycine, salicylic acid, benzoyl peroxide, N-acetyl-DL-methionine, terpineol, thymol, glycerin, retinol, retinyl palmitate, retinyl propionate, 4-ethyl resorcinol, 4-hexyl resorcinol, or a mixture thereof. In another embodiment and when used, such ingredients may make up (alone or collectively) from 0.0001 to 6%, or from 0.001 to 5%, or from 0.01 to 4% or from 0.01 to 3% or from 0.01 to 2% (or 0.1 to 1.5% or 0.1 to 1.25 or 0.1 to 4.3%) by weight of the wash composition.

In still another embodiment of the invention, the anionic surfactant used is a glycinate, taurate or a mixture thereof, the same being used with cocamidopropyl betaine that has 93.5 to 100% or from 95 to 100% or 100% of its hydrophobic portion being fatty acid and not triglyceride derived.

As noted herein, the wash comprises less than 0.065%, and preferably, less than 0.062%, and most preferably, less than 0.06% by weight or less ethylene glycol dilaurate based on total weight of the wash composition. The materials used, including the taurates incorporated in the wash compositions of the present invention, are preferably, not manufactured via a process that yields ethylene glycol dilaurate in excess. Taurates, for example, manufactured in the presence of excess fatty acid and ethylene glycol are not preferred. For illustration, should a defined amount of surfactant be included with enough ethylene glycol dilaurate impurity such that the end use wash composition has 0.065% or more ethylene glycol dilaurate, such surfactant is not desired for use in the wash composition of the invention.

When making the wash composition, the desired ingredients may be mixed with conventional apparatus under moderate shear and atmospheric conditions, with temperature being from 30 to 85° C. whereby shear continues until a homogeneous product is recovered.

The packaging for the wash composition typically is not limited as long as composition can be dispensed. In an embodiment of the invention, wash composition is sold in a pouch, bottle, jar, tube or canister. The packaging preferably is biodegradable and/or is prepared from recycled materials including post-consumer resins. Paper bottles are also suitable for use as are those that comprise bamboo. Other suitable formats include impregnating the wash composition in nonwovens like those having cotton, polyester or both or by providing the end use products in water soluble sachets such as those essentially consisting of polyvinyl alcohol.

In an embodiment of the invention, and surprisingly, the wash composition having from than 0.1 to 2% by weight PPAR agonist can be conveniently dispensed from a foam dispensing pump including one having a mesh pore or aperture size from 40 to 375 microns, or from 45 to 365 microns, or from 50 to 345 microns or from 70 to 335 microns, or from 90 to 315 microns, or from 90 to 125 microns. In another embodiment, such a foam dispensing pump will dispense wash composition at a volume from 0.4 to 3.7 cc, or from 0.5 to 3.6 cc, or from 0.6 to 3.5 cc, or from 0.6 to 3.2 cc, or from 0.6 to 1.6 cc, or from 0.6 to 1.2 cc.

When using the wash composition of the invention, the composition is often used with water at a composition to water weight ratio from 1:6 to 1:3 or from 1:5 to 1:4.5 or from 1:4.5 to 1:4, contacted with a surface, like skin, and rinsed off with water.

The Examples are provided to facilitate an understanding of the invention. The Examples are not intended to limit the scope of the claimed invention.

### Example I

Wash compositions (Samples 1 to 7) as defined in Table I were made with the ingredients listed in the Table. All ingredients were combined by mixing with moderate shear. Mixing temperature was stabilized between 55 to 80° C. and pressure was atmospheric. The resulting wash concentration was cooled to about 35° C. and pH was adjusted to 6.8 to 7.1. The percents shown in the Table and referred to in this Example are percents by weight. Viscosity was taken at 25° C. with a Discovery HR-2 Rheometer using sand blasted plates having a 1000-micron gap and a shear rate of 10⁻¹ s⁻¹. For the compositions made according to the present invention, viscosity was surprisingly stable at about 13 mPa-s for 2 months when stored at 22° C. All numbers in the table are percent by weight of the composition.

TABLE

| | Sample^{C} | Sample^{P} | Sample^{F} | Sample^{P} | Sample^{F} | Sample^{P} | Sample^{F} |
|---|---|---|---|---|---|---|---|
| | 1 | 2 | 3 | 4 | 5 | 6 | 7 |
| Ingredient | Wt. % | Wt. % | Wt. % | Wt. % | Wt. % | Wt. % | Wt. % |
| Water | Balance | Balance | Balance | Balance | Balance | Balance | Balance |
| Sodium Cocoyl Glycinate | | 4.3 | 4.3 | | | | |
| Sodium Lauroyl Glycinate | 2.2 | | | | | | |
| SLS | | | | 4.3 | | | |
| Na Methyl Lauroyl Taurateⁱ | | | | | 4.3 | | |
| Na Methyl Lauroyl Taurateⁱⁱ | | | | | | 4.3 | |
| Na Lauroyl Isethionate | | | | | | | 4.3 |
| C₁₂-triglyceride propyl | 3.8 | | 7.7 | | | | |
| betaine | | | | | | | |
| Cocamidopropyl Betaine, | | 7.7 | | 7.7 | 7.7 | 7.7 | 7.7 |
| fatty acid derived | | | | | | | |
| Stearic Acid | 0.25 | 0.25 | 0.25 | 0.25 | 0.25 | 0.25 | 0.25 |
| Lauric Acid | 0.10 | 0.10 | 0.10 | 0.10 | 0.10 | 0.10 | 0.10 |
| 12-Hydroxystearic Acid | 0.1 | 0.25 | 0.25 | 0.25 | 0.25 | 0.25 | 0.25 |
| Glycerin | 5.0 | 1.0 | 1.0 | 1.0 | 1.0 | 1.0 | 1.0 |
| Phenoxyethanol | 0.6 | 0.6 | 0.6 | 0.6 | 0.6 | 0.6 | 0.6 |
| IPBC | 0.01 | 0.01 | 0.01 | 0.01 | 0.01 | 0.01 | 0.01 |
| PPG-7 | 0.5 | 0.5 | 0.5 | 0.5 | 0.5 | 0.5 | 0.5 |
| Sodium Hydroxide | 0.01 | 0.01 | 0.01 | 0.01 | 0.01 | 0.01 | 0.01 |
| Citric Acid | 0.4 | 0.05 | 0.05 | 0.05 | 0.05 | 0.05 | 0.05 |
| Sodium Gluconate | 0.05 | 0.05 | 0.05 | 0.05 | 0.05 | 0.05 | 0.05 |
| Fragrance | 0.9 | 0.9 | 0.9 | 0.9 | 0.9 | 0.9 | 0.9 |

The superscripts, ^{C}; ^{P}; and ^{F} denote control formula, passing formulae consistent with the invention and failed formula, not made consistent with the invention, respectively. Superscript ⁱ indicates sodium methyl lauroyl taurate provided with enough ethylene glycol dilaurate impurity to yield a wash composition with more than 0.065% by weight of the impurity. Superscript ⁱⁱ indicates sodium methyl lauroyl taurate provided with less ethylene glycol dilaurate impurity to yield a wash composition with more than 0.065% by weight of the impurity.

Wash compositions were made consistent with the present invention, Samples 2, 4 and 6, surprisingly were clear and comprised 0.25% by weight 12-hydroxystearic acid. The compositions made according to the invention were stable, lathered well and rinsed off easily with water, displaying traditional characteristics typically enjoyed by consumers as concluded by trained panelists. No discoloration, cloudiness or creaminess was detected with visual inspection. The wash compositions of Samples 2, 4 and 6 were pumpable in a foam pump dispenser having a mesh size of 100 microns.

Sample 1, the control formula was not suitable to contain more than 0.1% by weight 12-hydroxystearic acid. The PPAR agonist at amounts that exceeded 0.1% by weight resulted in a cloudy, hazy, opaque wash composition that was not suitable to pump in a foam pump dispenser having a mesh size of 100 microns.

Samples 3, 5 and 7, not made consistent with the invention, were cloudy and opaque.

### Example II

In this Example three samples were made, Samples 8, 9 and 10, via a method similar to the one described in Example I. The Samples were made consistent with Sample 7 in the Table except that Samples 8, 9, and 10 had 1%, 2% and 3% sodium lauroyl isethionate, respectively, and not 4.3% sodium lauroyl isethionate as indicated in Sample 7.

Surprisingly, Samples 8, 9 and 10, made consistent with the present invention, were clear and comprised 0.25% by weight 12-hydroxystearic acid. The compositions made according to the invention were stable, lathered well and rinsed off easily with water, displaying traditional characteristics typically enjoyed by consumers as concluded by trained panelists. No discoloration, cloudiness or creaminess was detected with visual inspection and the wash compositions of Samples 8, 9 and 10 were pumpable in a foam pump dispenser having a mesh size of 100 microns. Sample 7, was not clear, appeared hazy or creamy and was not pumpable.

What is claimed is:

1. A wash composition comprising:
a) from 4 to 18% by weight total surfactant, the surfactant comprising anionic surfactant, and amphoteric surfactant wherein 50 to 90% by weight of total surfactant in the wash composition is amphoteric surfactant;
b) preservative;
c) from 0.1 to 2% by weight of an activator of a peroxisome proliferator activated receptor based on total weight of the wash composition;
d) alcohol; and
e) water,
wherein the composition:
i) is transparent;
ii) has a pH from 5.65 to 8.5; and
iii) comprises less than 0.065%, and preferably, less than 0.062%, and most preferably, less than 0.06%, by weight ethylene glycol dilaurate based on total weight of the wash composition,
the amphoteric surfactant comprises 25% or less by weight hydrophobic portion derived from triglyceride based on total weight of hydrophobic portion present on the amphoteric surfactant and with the proviso that if the anionic surfactant comprises acyl isethionate, total weight of acyl isethionate included does not exceed 3.6% by weight of the total weight of the wash composition.

2. The wash composition according to claim 1 wherein the activator of a peroxisome proliferator activated receptor is 12-hydroxystearic acid.

3. The wash composition according to claim 1 wherein the preservative comprises phenoxyethanol.

4. The wash composition according to claim 1 wherein the composition comprises less than 1% by weight sulfate-based surfactant, and the amphoteric surfactant used comprises 0.0% by weight hydrophobic portion derived from triglyceride based on total weight of hydrophobic portion present on the amphoteric surfactant.

5. The wash composition according to claim 1 wherein the composition does not comprise an acrylate-based thickener and a hydantoin.

6. The wash composition according to claim 1 wherein the activator of a peroxisome proliferator activated receptor composition comprises 10-hydroxystearic acid, pertoselinic acid, conjugated linoleic acid, 12-hydroxy-9-cis-octadecanoic acid, palmitoleic acid, stearolic acid or a mixture thereof.

7. The wash composition according to claim 1 wherein the wash composition is substantially free of isothiazolinone, phthalate, soap, dioxane and silicone oil.

8. The wash composition according to any of the preceding claims wherein the wash composition further comprises thiamidol, oleyl adapalenate, hyaluronic acid, 10-hydroxystearic acid, mandelic acid, lactic acid, caffeine, tretinoin, epigallocatechin gallate, adapalene, 2-mecaptonicotinoyl glycine, salicylic acid, benzoyl peroxide, terpineol, thymol, glycerin, retinol, retinyl palmitate, retinyl propionate, 4-ethyl resorcinol, 4-hexyl resorcinol, or a mixture thereof.

9. The wash composition according to claim 1 wherein the composition further comprises a fatty acid.

10. The wash composition according to claim 1 wherein the composition has a viscosity from 1 to 1,100 mPa-s.

11. The wash composition according to claim 1 wherein the anionic surfactant comprises at least one of a glycinate, taurate, isethionate, glutamate, succinate, sulfonate, sarcosinate, alaninate, and the amphoteric surfactant comprises a betaine.

12. The wash composition according to claim 1 wherein the composition is pumpable from a foam pump dispenser.

13. A method for washing a surface comprising, in no particular order, the steps of contacting a surface with the wash composition according to claim 1 and water, and rinsing the composition off with water.

14. The method for washing a surface according to claim 13 wherein the surface is skin or hair.

15. The method according to claim 14 wherein the wash composition has a viscosity from 1 to 45 mPa-s and the wash composition is pumped from a foam dispensing pump having a mesh pore size from 40 to 375 microns.
